(19) United States
(12) Patent Application Publication
(10) Pub. No.: US 2004/0034338 A1
(43) Pub. Date: Feb. 19, 2004

(54) Drug delivery devices and methods

(75) Inventors: Christopher A. Thierfelder, Minneapolis, MN (US); Robert C. Grant, New Hope, MN (US)

Correspondence Address: Attention: Jeffrey J. Hohenshell, AMS Research Corporation, 10700 Bren Road West, Minnetonka, MN, 55343, US

(73) Assignee: AMS Research Corporation

(21) Appl. No.: 10/636,182

(22) Filed: Aug. 7, 2003

Related U.S. Application Data
(60) Provisional application No. 60/401,934, filed on Aug. 7, 2002.

(51) Int. Cl. A61K 9/22

(52) U.S. Cl. 604/891.1

(57) ABSTRACT

Implantable drug delivery devices and components are disclosed. The devices may be implanted in a wide variety of locations in a patient's anatomy. Novel methods for treating a variety of disorders are also disclosed.

### CROSS REFERENCE TO RELATED APPLICATION

The present application claims priority to U.S. Provisional Application Serial No. 60/401,934, filed Aug. 7, 2002. The entire contents of that patent application are herein incorporated by reference.

### BACKGROUND

The design of an implantable drug delivery device faces many hurdles. One significant hurdle is the body's foreign body response. Over time, this response will tend to occlude a drug delivery opening in an implantable drug delivery device. For example, if the device includes a catheter with an opening for delivering the drug, the foreign body response creates a fibrous occlusion that can adversely affect drug delivery, dose reproducibility and the overall function of the device.

Another problem confronting designers of implantable drug delivery devices is drug storage. Not all drugs are stable at body temperature. For example, prostaglandin E1 {also known as Alprostadil and Caverject (Pharmacia, Inc.) (hereafter PGE1)} is the only drug approved by the U.S. Food and Drug Administration for use in injection therapy for erectile dysfunction. However, PGE 1 is reactive and unstable, and cannot be stored in its biologically active form at body temperature. One of PGE1 's drawbacks is that it must be stored at very cold temperatures to maintain potency.

Some patients experience discomfort with injection (or administration by transepithelial absorption) of a substance for treating erectile dysfunction. Such procedures can have variable effects. Some patients complain that such therapies lack desirable spontaneity.

Implantable devices for delivering drugs for treating erectile dysfunction are disclosed in U.S. Pat. Nos. 4,766,889 and 5,518,499 and published U.S. Pat. application Ser. No. 2001/0041824-A1. Despite the presence of implantable drug delivery devices in the literature, there are no large scale commercial embodiments of an implantable drug delivery device for treating erectile dysfunction.

The polymer poly(glycine-valine-glycine-valine-proline) has been used in abdominal surgeries to reduce cell attachments at wound site and to limit abdominal adhesion formation. See Sakiyama-Elbert, et al Functional Biomaterials: Design of Novel Biomaterials, Ann. Rev. Mat. Res. Aug 2001, Vol. 31, pp. 183-201.

Microfluidic technology is currently used in in vitro fertilization clinics in large-animal veterinary hospitals to handle embryos. See Hickman, D. L., D. J. Beebe, S. L. Rodriguez-Zas and M. B. Wheeler, Comparison of static and dynamic medium environments for the culture of preimplantation mouse embryos, J. Comparative Medicine, April 2002, 52:122-126., Beebe, D. J., M. B. Wheeler, H. C. Zeringue, E. Walters and S. Raty, Microfluidic technology for assisted reproduction, Theriogenology, Vol. 57, No. 1, pp. 125-135, 2002, Wheeler, M. B., D. J. Beebe, E. M. Walters, S. Raty, Microfluidic Technology for In Vitro Embryo Production IEEE-EMBS-MMMB Conference 2002, pp104-108. Handling embryos requires significant external equipment to maintain an appropriate environment of the embryos to grow. This can include, but is not limited to, heaters, syringe pumps, electronic valves, growth chambers, indexing units, sorting units, and a sterile water or saline supply.

### SUMMARY OF THE INVENTION

The present invention is directed to implantable drug delivery devices, components and procedures. More particularly, the present invention comprises an implantable drug delivery device for on-demand treatment of erectile dysfunction.

In one aspect, the present invention comprises an implantable drug delivery system. The system may include any suitable drug and may be used to treat a wide variety of disorders. In a preferred embodiment, the system is adapted to deliver prostaglandin E1 for treating erectile dysfunction. The system comprises a housing suitable for implantation in a patient; storage means (e.g. a storage chamber or compartment) for storing a quantity of drug (e.g. in a dry powder), metering means for metering a predetermined, effective amount of the drug; and delivery means for delivering an effective amount of the drug to a patient to treat a disorder.

The storage means preferably comprises a plurality of storage compartments. In one embodiment, the metering means comprises a plurality of micro-channels capable of communicating with the storage compartments, a mixing chamber, and valve means capable of being opened to afford fluid communication with the storage compartments. In a preferred embodiment, the system includes indexing means for affording indexed communication between the mixing chamber and a micro-channel. The system preferably includes a catheter with drug delivery ports that are sized and shaped to be implanted in a corporal body region of a patient. The catheter may optionally include a coating (poly(glycine-valine-glycine-valine-proline)). Alternatively, the device may include storage means for a substance (e.g. biodegradable polymer) for resisting fibrous occlusion of the drug delivery port.

In a preferred embodiment, the delivery means comprises a pump. Power for the pump may be provided by the patient (e.g. during actuation) or it may have a dedicated power supply (e.g. a battery).

The valve means may comprise a plurality of different embodiments as well. The valves can be controlled through a variety of means. Some embodiments do not need to be manipulated by hand. Indexing is preferably automatic. Metering and delivery require only very small amounts of fluid. As a result, fluid can be drawn from either the body/external environment, or from a very small reservoir

In another aspect, the invention comprises a system with storage means for storing a drug, metering means for metering a predetermined, effective amount of the drug; and delivery means for delivering an effective amount of the drug to a patient to treat a disorder. The delivery means comprises a catheter having a plurality of drug delivery ports. The drug delivery ports are movable between an open position to deliver the drug to the patient, and a closed position. In this embodiment, the system includes drug delivery path preservation means for resisting fibrous occlusion of the drug delivery ports. The drug delivery path preservation means may comprise poly(glycine-valine-glycine-valine-proline) associated with the catheter. Alternatively, it may comprise a means for delivering a substance for resisting fibrous occlusions through drug delivery ports in a catheter. In yet other embodiments, it may comprise a fluid or film on the catheter.

In one embodiment, the device is self-contained. It does not require temperature regulation. It does not need significant pressure. In some embodiments, several micro pumps can be contained within the device. Some embodiments do not require extensive patient monitoring or patient involvement other than activation. It will be appreciated that some embodiments do not require careful loading of active components.

Preferably, the devices are small to facilitate implantation.

In another aspect, the present invention comprises a method of treating erectile dysfunction. The method comprises the steps of: implanting a supply of prostaglandin E1 in the body in a device capable of releasing a dose on demand, and thereafter treating the erectile dysfunction by releasing an effective amount of prostaglandin E1 on demand of the patient. Optionally, the method may include the step of resisting the chances of an overdose of the prostaglandin E1 by preventing actuation of the device outside predetermined parameters.

### BRIEF DESCRIPTION OF THE DRAWINGS

Other features and advantages of the present invention will be seen as the following description of particular embodiments progresses in conjunction with the drawings, in which:

FIG. 1 is a schematic illustration of one option for placing one embodiment of an implantable drug delivery device according to the present invention;

FIG. 2 is a perspective view of components of one embodiment of implantable drug delivery device according to one embodiment of the present invention,

FIG. 3 is a perspective view of additional components of the embodiment of implantable drug delivery device shown in FIG. 2;

FIG. 4 is a side view of one component of another implantable drug delivery device according to the present invention;

FIG. 5 is a schematic cross-sectional view of a self closing drug ejection port of the device of FIG. 4, illustrating pressure building with a plurality of arrows;

FIG. 6 is a schematic cross-sectional view of the drug ejection port of FIG. 5 after pressure has built to a point where it temporarily opens the port to deliver drug;

FIG. 7 is a perspective view of a component of an implantable drug delivery device according to another aspect of the present invention, with a portion of the component enlarged to illustrate details; and

FIG. 8 is a view of the component of FIG. 7 which uses an arrow to depict one element being delivered to the body.

### DETAILED DESCRIPTION

The following description is meant to be illustrative only and not limiting. Other embodiments of this invention will be apparent to those of ordinary skill in the art in view of this description.

The present invention is directed to novel implantable drug delivery devices, components thereof and methods of treating disorders. The devices are suitable for delivering any drug that can be stored over time at body temperature. The drug may be stored in a ready to deliver condition. Alternatively, it may be stored in a precursor condition (e.g. dry powder form) and mixed with another component (e.g. a liquid) just prior to delivery. The drug may be stored in any suitable condition such as in solution, in dry powder, as a fluid or gel.

The implantable devices according to the present invention may deliver any substance capable of providing a desirable therapeutic effect. Suitable substances include, but are not limited to analgesics, hormones, anti-inflammatants, anti-fibrotic agents, and antibiotics. Substances that are believed to be suitable for delivery include prostaglandin E1, PGE1/alpha-cyclodextrin complexes, PGE1/beta-cyclodextrin complexes, phentolamine, papaverine, sodium nitroprusside, nitrous oxide, any vasodilator, any of the above components with any known or unknown excipient. The devices may also be used to treat any disorder such as erectile dysfunction, infections (e.g. bone infections), diabetes, etc.

Preferably, the drugs are stored at body temperature for extended periods of time (e.g. 6 months). PGE1 can be stabilized as a 1:1 complex with the carbohydrate Beta-cyclodextrin. The beta-cyclodextrin molecule is essentially six glucose molecules joined to form a ring. In 3d-space, this ring orients itself into a hollow cone. During processing, a single PGE1 molecule is inserted into this cone. Complexation stabilizes the PGE1 from heat at increased temperatures for a much longer duration. The sugar cone is extremely stable. This could allow a dry base product to remain stable at 37° C. for up to a year, and a liquid based product to remain stable for up to 90 days, yet the PGE1 is readily released in solution and exhibits full biological activity. See, Yamamoto, et al, Improvement of Stability and Dissolution of Prostaglandin E1 by Maltosyl-B-Cyclodextrin in Lyophilized Formulation, Chem. Pharm. Bull. 40(3) 747-751 (1992). Studies have shown that this PGE1-beta-cyclodextrin complex functions very well and slows the degradation process such that well over 50% of the product is available after 3 months of body-temperature aging. For comparison, some physicians have suggested that no more than 30% of PGE1 (sold under the name Caverject, available from Pfizer Corp.) needs to be chemically available for fair results. Caverject degrades to below 30% with 48 hours of being exposed to body temperature.

The excipient beta-Cyclodextrin is available commercially under the trade name Captisol, from CyDex in Kansas City, Mo. The U.S. Food and Drug administration has approved the use of Captisol in a variety of intravenous therapies with other drugs. It is also available in its pure form from Cyclolab (Budapest, Hungary), and in various polymeric forms such as those available from Insert Therapeutics (Pasadena, Calif.).

In another embodiment, another complex sugar molecule may be used to stabilize a highly reactive drug. For example, PGE1 (Alprostadil) can be stabilized if complexed with maltosyl-beta-cyclodextrin (G₂-b-CyD).

In another embodiment, PGE1 is emulsified with a light oil and deposited in the implantable delivery device that includes a pump. The pump also contains a chamber of solvent that mixes with PGE1 emulsion on (patient) demand. The solvent breaks the emulsion and the PGE1 can be delivered to the corpora.

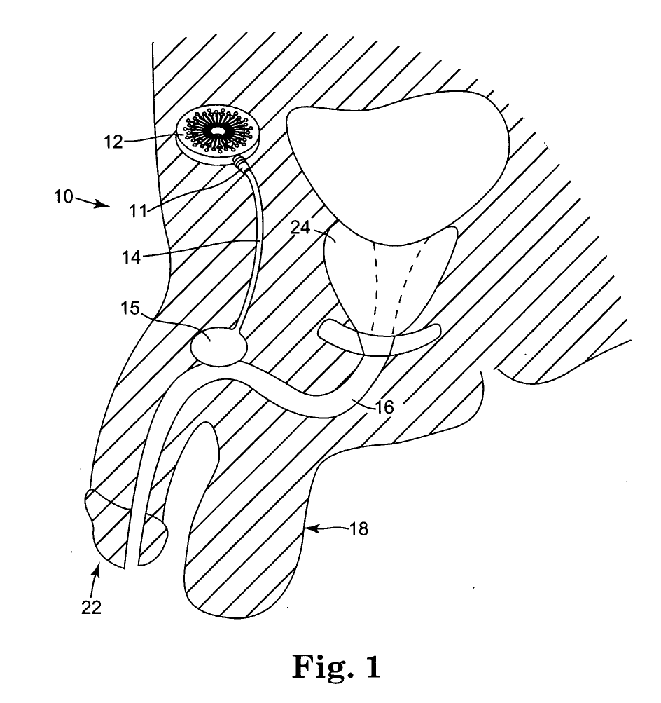

In a preferred embodiment, the present invention comprises an implantable, on-demand, drug-delivery system affording on-site delivery of erectile dysfunction treatment drugs without transcutaneous injection into the penis. Referring to FIGS. 1-3, the delivery port/end of a catheter 14 is inserted in a suitable location such as directly into the corpora 15, or into the dorsal vein of the penis. On the other end, a device 10 including reservoirs or storage chambers 36, pump (alternatively a sub-cutaneous septum, not shown), and other components (described more fully below) is placed just below the surface of the skin (preferably in the abdomen, but alternatively in other areas such as the scrotum 18). The device shown in FIG. 1 is implanted in a male patient with the urethra 16 and prostate 24 shown out of scale. This embodiment is particularly suitable for treating erectile dysfunction. In devices where a septum is utilized, it is placed to receive the drug. This device allows the patient to inject the drug more comfortably than previously allowed into the penis.

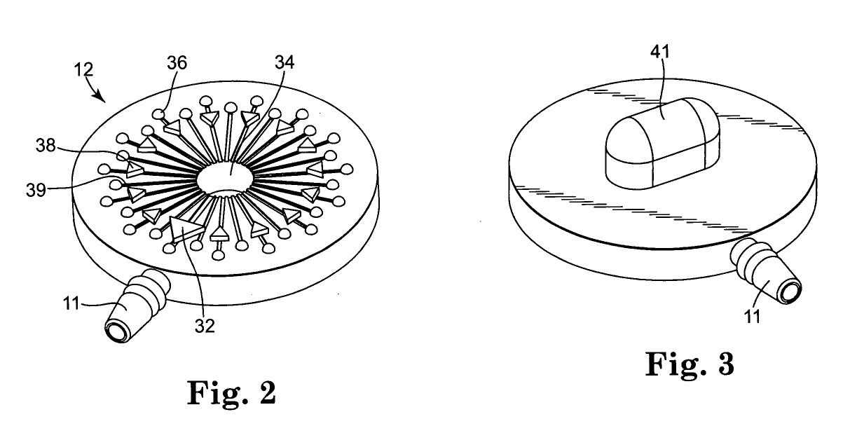

A preferred embodiment of device 12 is shown in FIGS. 2 and 3. Single doses of dry drug (in powder form) may be stored in a plurality of storage chambers such as the raised blisters 36. Each blister 36 is sealed during manufacture, and preferably has an internal moisture content near zero when the drug is in dry powder form. The blister 36 may or may not be made of a charge-sensitive, semi-permeable membrane.

Fluid management within the devices of the present invention may exploit pressure differentials (e.g. supplied by a pump) or the tendency of a fluid to flow in a microchannel, or combinations thereof. Notably, some microspheres suitable for delivering drugs in embodiments of the present invention are substantially the size of embryos. Microchannel handling of embryos and fluids are disclosed in U.S. Pat. No. 6,193,647, published U.S. Pat. application Ser. No. 2003/0077836-A1; Eddington et al., An organic self-regulating microfluidic system, Lab on a Chip, 2001, 1, 96-99; Beebe et al, Functional hydrogel structures for autonomous flow control inside microfluidic channels, Nature, Vol. 404 (Apr. 6, 2000) Pp. 588-590; and Yu et al., Responsive Biomimetic hydrogel valve for microfluidics, Applied Physics Letters, Vol. 78, No. 17 (April 2001) Pps. 2589-2591.

Pumps may be self contained in the devices with an internal power source. Alternatively, the device may include pumps powered by the patient (e.g. with a membrane that is squeezed or otherwise manipulated).

In FIG. 2, the blister 36 is connected to the main fluid pathways by a micro-channel 39 which may, for example, be no greater than 500 micron (0.5 mm) in diameter. By rotating, a main mixing chamber 34 comes into fluid communication with a blister 36. As the device 12 is used, the patient indexes from blister to blister, taking the medicament from each in turn.

Upon activation, the blister 36 releases the drug in any suitable fashion. For example, a battery 41 (FIG. 3) can charge the blister 36, causing the membrane to allow fluid from the body to enter, and turn the solid powder into an aqueous solution, or at minimum, a suspension.

Alternatively a fluid may be stored in the device 12 for subsequent combination with the medicament. In this embodiment, an input valve is opened by a battery charge, and fluid from a reservoir on the underside of the device 12 flows into the blister, to the same effect.

After a short delay, valves 38 open, allowing the solution to travel down the micro channel. Because the total volume of the fluid required is very small (micro liters), the input from either the reservoir or the body causes enough pressure differential to force the fluid down the channel via a pressure differential, capillary action or both. The valves may be hydrogel-based valves, and can be activated by electrical charge, chemical interaction with the body, fluid pressure, chemical interaction with the drug, pH changes in the environment or ultraviolet wave interaction.

Input of the solution into the mixing chamber 34 brings the solution into a small pumping area, where the drug is further mixed, and sent out the main output tube 11. Optional main blocking valve 32, allows this final dose to enter the body. This valve may or may not be controlled by a higher-level timing circuit which can be programmed to allow dosage only at safe intervals. At this point, the mixing pump will drive the solution down the catheter 14 and into the corpora 15.

The system 10 of FIG. 1 ends in a catheter 14. The catheter 14 terminates with at least one drug delivery port. It is important for the catheter 14 and port to be kept clean (free from cellular material), to allow for the device to deliver drugs at the widest possible intervals and with a repeatable effective amount. The material from which the catheter is constructed could be any suitable implantable material. For example, the material can be copolymer such as an extruded Polyurethane-Silicone copolymer called PurSil, available commercially from The Polymer Technology Group (Berkeley, Calif.).

A novel copolymer using PurSil as a substrate may also be used. The new copolymer adds polyethylene oxide groups to the surface of the material, which helps mask the material from the body's foreign-body reaction. In studies in animals, and with human whole blood, this material potentially remains free of obstructions after long term implantation studies (per ISO 10993).

In another embodiment, a coating may be utilized. For example, the coating produced by Biocompatibles Ltd. UK, which incorporates phosphorylcholine (a primary component of cell membranes) may also be used. Red blood cells have a unique signature which identify them as red blood cells, and therefore not to be attacked by the body as a foreign agent. While all cells, tissues, and organs have such a signature, the red blood cell signature is significant in its simplicity, and ability to be synthesized. Red blood cells do not have typical cell attachment sites, which proteins look for when identifying cells. Instead, they rely on a phospholipid layer, which produces a uniform chemical signature and charge to identify themselves as part of the body.

This phospholipid layer can be synthesized in a molecule known as a phosphorylcholine headgroup. When synthesized in the same spatial configuration as the RBC surface and applied to a substrate, the body has been shown to ignore the implant, and lessen or eliminate its response.

In some embodiments, the catheter coating can attract water molecules to the surface, bind the water tightly, and form a water-barrier. When neutrophils (or bacteria, or other cells) are in the vicinity, they do not perceive the device as a foreign body, and continue moving. This chemical effect, as well as having a mechanically soft catheter is believed to help reduce or eliminate the fibrous encapsulation.

PEG (polyethylene glycol—the primary component of EtO) molecules have the capability of retarding the cellular adhesion that precedes fibrous capsule formation. They do this by creating a “shield” of water molecules, which inhibit protein adhesion to the surface. The spatial alignment of the PEG molecules help them act as a shield; and they are a coating, not integral components of the underlying material

In another embodiment, the catheter may be constructed from a biomimetic (biology-mimicking) material. By using a biomimetic material, the body can be fooled into reacting as if the implant is another piece of tissue, thereby reducing or eliminating the natural response to foreign materials. In this embodiment, the catheter is constructed from a silicone, first formulated to have similar mechanical properties to biologic tissue. This affords an initial reduction in the tissue's response, as the fibrous capsule response is heightened by rigidity of material. The silicone also has another component which will allow it to elute an oily coating over the surface. The coating may be an oil (fatty alcohol). Due to the aqueous nature of the body's chemistry, it has limited solubility in the body. The fluid nature of this coating prevents formation of stable cell adhesions, thereby limiting encapsulation. In this embodiment, the oil-elution properties of a specially formulated silicone, processed by the Xiomateria group in Belfast, North Ireland are exploited. See Gorman S. P., Tunney M. M., Keane P. F., Van Bladel K., Bley B. (1998), Characterisation and assessment of a novel poly(ethylene oxide)/polyurethane composite hydrogel (Aquavene) as a ureteral stent biomaterial, J. Biomed. Mater. Res. 39, 642-650.

In another embodiment, one or more of the following methods may be exploited to reduce or eliminate the fibrous encapsulation of indwelling implants. Each of these methods can be used either alone or with a self-renewing coating, such as that provided by Sil-Xtra silicone, or, a separate surface bound coating can be used with these agents and applied separately.

CTGF Blocker: Connective tissue growth factor (CTGF) is one cytokine which triggers and maintains fibrosis. This factor acts on the cells which produce it, thereby causing the proliferation of fibrous tissue. By blocking this cytokine using human monoclonal antibodies or other inhibitors, the process of fibrotic encapsulation will not initiate or proliferate. See Brigstock DR., The connective tissue growth factor/cysteine-rich 61/nephroblastoma overexpressed (CCN) family, Endocr Rev. 1999 April; 20(2):189-206. Grotendorst GR Connective tissue growth factor: a mediator of TGF-beta action on fibroblasts, Cytokine Growth Factor Rev. 1997 Sep; 8(3):171-9.

C-Proteinase Blocker: C-Proteinase converts procollagen to fibrillar collagen, the primary extracellular matrix component of the fibrous capsule. By blocking this enzyme, the fibrillar collagen cannot form and will not create the capsule.

Prolyl Hydroxylase: This is an enzyme required to create procollagen (the precursor to the fibrillar collagen that makes the capsule). By blocking this enzyme using small molecule inhibitors, the process is halted, and the capsule matrix is not formed.

In another embodiment, poly(GVGVP) (GVGVP=glycine-valine-glycine-valine-proline) is used to reduce cell attachment at the wound site and also to limit adhesion formation. It is generally accepted that the hydrophobic nature of the oligopeptide serves to block cell adhesion.

GVGVP has been used successfully to block fibrous adhesion, and has also been grafted to silicone rubber using photochemical immobilization (Surmodics, Inc.) In that instance, the silicone with GVGVP has been implanted in Sprague-Dawley rats and showed a score of 0-1 when rating the quality of fibrous encapsulation (0=none, +1=minimal, +2=mild, +3=moderate, +4=extensive). For reference, the uncoated control group scored 2-3. While some cell adhesion was noticed, it is reasonable to assume the limited cell adhesion on the surface will not significantly inhibit drug delivery from a catheter.

In another embodiment, the fibrous capsule formation is reduced or completely eliminated through the use of polylacticglycolic acid (PLGA) microspheres to deliver dexamethasone to the delivery site. Dexamethasone is a corticosteroid anti-inflammatory and has been shown to be effective in minimizing—and in some cases, eliminating—the inflammatory response and subsequent fibrotic encapsulation. At the time of implantation, the physician could also inject the site with PLGA microspheres prepared with dexamethasone. Based on the formulation, the spheres could maintain a fibrous-free zone for a period of time (e.g. a month), for example, the duration of a supply of erectile dysfunction treatment. Every 30 days, when the patient returns to the physician (e.g. to refill a drug reservoir in embodiments with refillable reservoirs), the physician could begin by using the drug delivery unit to deliver another 30-day dosage of microspheres to the injection site. This facilitates maintenance of the fibrous-tissue-free zone, without additional inconvenience to the patient.

In another embodiment, the solution for maintaining the drug delivery port free of encapsulation may be self contained within the device so that it can be periodically actuated to keep the port free of blockage.

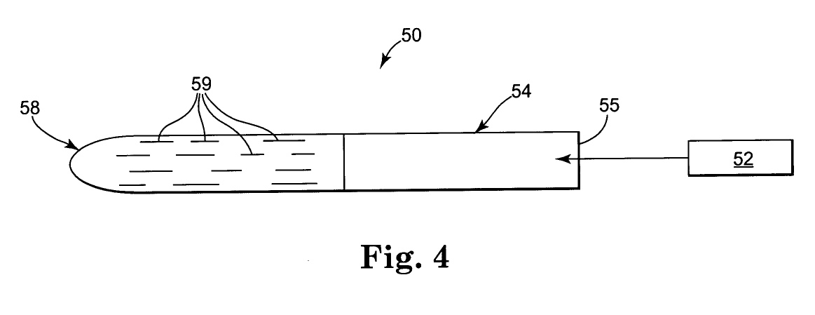

Referring now to FIGS. 4 through 6, there is shown a catheter 50 having a plurality of drug delivery ports 59. The catheter has a main body 54 and a drug delivery portion 58. The proximal end 55 of the catheter 50 is in fluid communication with reservoir and metering means 52. The reservoir and metering means may be any suitable means, such as those described above, which may also include optional pumps, overdosage prevention, and other features.

Referring now to FIGS. 4 through 6, there is shown a catheter 50 having a plurality of drug delivery ports 59. The catheter has a main body 54 and a drug delivery portion 58. A single drug delivery portion is illustrated in FIG. 4. The catheter may also contain multiple drug delivery segments separated by nonporous segments to deliver drug simultaneously at several points along the catheter. The proximal end 55 of the catheter 50 is in fluid communication with reservoir and metering means 52. The reservoir and metering means may be any suitable means, such as those described above, which may also include optional pumps, overdosage prevention, and other features.

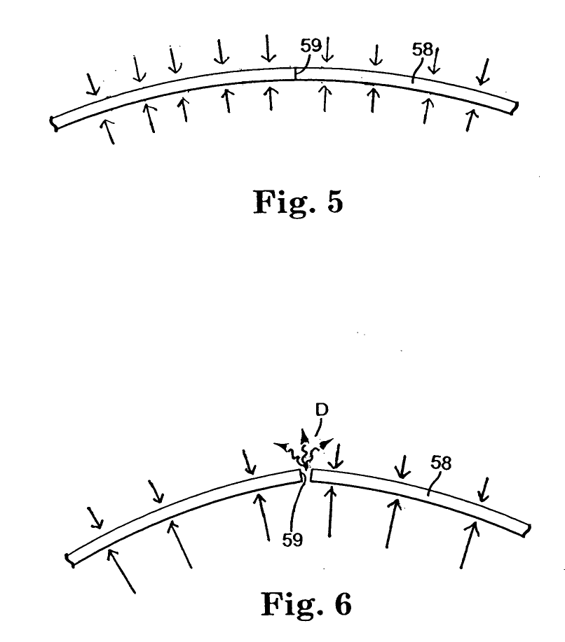

Referring to FIGS. 5 and 6, the drug delivery ports are movable between an open position (FIG. 6) to deliver the drug to the patient, and a closed position (FIG. 5). The wall of the drug delivery portion 58 is comprised of an elastomer. In the absence of interior pressure from the drug delivery pump the drug delivery ports are maintained in the closed position (FIG. 5). This is accomplished by careful control of the physical properties of the elastomer, the wall thickness, and the length of the drug delivery ports. When the drug delivery pump increases fluid pressure within the catheter lumen, the drug delivery tip expands slightly allowing release of drug-containing fluid through the ports (FIG. 6). The ports close automatically as internal pressure is relieved, thereby preventing entry of fluid or cells from the surrounding tissue. The outer surfaces of the drug delivery sections may be coated as described above to inhibit tissue attachment. At the same time the nonporous sections may be treated to permit or enhance tissue attachment for catheter fixation.

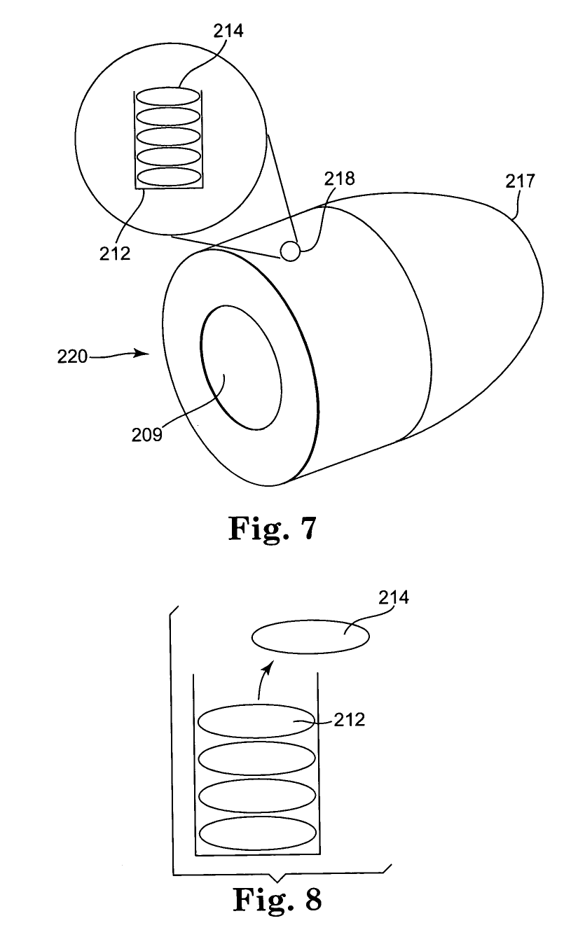

Referring now to FIGS. 7 and 8, there is shown another embodiment of catheter according to the present invention, the catheter 220 has a distal tip 217 and an internal passageway 209. In this embodiment, the tip is made from stainless steel, tantalum, polymer, or any material that is biocompatable and any of the following: 1.) machineable, 2.) porous, or 3.) moldable. Here, the device takes the form of a double-lumen “bullet” shape, with thousands of micro-holes 218 across the surface. In these holes are stacked many tiny discs 212 of an anti-inflammatory agent, ultra-concentrated, and dissolvable in aqueous solution. When the catheter with this tip is implanted, the outermost discs 214 slowly dissolve or become dissociated from the tip, retarding the healing or fibroid producing process and creating a zone around the delivery site free of fibrous encapsulation. The tip is then unfettered to release the drug into the delivery site.

The devices according to the present invention are capable of construction in a wide variety of sizes and shapes. In the microchannel embodiments, the entire device is preferably made as a circle with the diameter of a half-dollar, and roughly 0.5-cm thick.

All patents, patent applications, and publications cited herein are hereby incorporated by reference in their entirety as if individually incorporated.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. An implantable drug delivery system comprising:
a housing suitable for implantation in a patient;
storage means for storing a quantity of drug in a dry powder form,
metering means for metering a predetermined, effective amount of the drug; and
delivery means for delivering an effective amount of the drug to a patient to treat a disorder.

2. An implantable drug delivery system according to claim 1 wherein the delivery means comprises:
a catheter having a plurality of drug delivery ports, the drug delivery ports being movable between an open position to deliver the drug to the patient, and a closed position; and
drug delivery path preservation means for resisting fibrous occlusion of the drug delivery ports.

3. An implantable drug delivery system comprising:
a housing suitable for implantation in a patient,
a storage chamber for storing a quantity of drug,
metering means for metering a predetermined, effective amount of the drug; and
delivery means for delivering an effective amount of the drug to a patient to treat a disorder.

4. An implantable drug delivery system according to claim 3 wherein the storage means
comprises a plurality of storage compartments,
the metering means comprises a plurality of micro-channels capable of communicating with the storage compartments, a mixing chamber, and valve means capable of being opened to afford fluid communication with the storage compartments.

5. An implantable drug delivery system according to claim 4 further comprising indexing means for affording indexed communication between the mixing chamber and a micro-channel.

6. An implantable drug delivery system according to claim 3 wherein the drug comprises prostaglandin E1.

7. An implantable drug delivery system according to claim 3 wherein the system includes
a catheter with drug delivery ports that are sized and shaped to be implanted in a corporal body region of the patient.

8. An implantable drug delivery system according to claim 7 wherein the catheter includes a coating of poly(glycine-valine-glycine-valine-proline).

9. An implantable drug delivery system according to claim 3 wherein the device includes a drug delivery port, and
storage means for a substance for resisting fibrous occlusion of the drug delivery port.

10. An implantable drug delivery system according to claim 9 wherein the substance for resisting fibrous occlusion comprises a biodegradable polymer.

11. An implantable drug delivery system according to claim 3 wherein the delivery means comprises a pump.

12. An implantable drug delivery system according to claim 4 wherein the valve means are battery powered.

13. An implantable drug delivery system comprising:
storage means for storing a drug,
metering means for metering a predetermined, effective amount of the drug;
delivery means for delivering an effective amount of the drug to a patient to treat a disorder, the delivery means comprising:
a catheter having a plurality of drug delivery ports, the drug delivery ports being movable between an open position to deliver the drug to the patient, and a closed position; and
drug delivery path preservation means for resisting fibrous occlusion of the drug delivery ports.

14. An implantable drug delivery system according to claim 13, wherein the drug delivery path preservation means comprises poly(glycine-valine-glycine-valine-proline) associated with the catheter.

15. A drug delivery system according to claim 13, wherein the catheter has a longitudinal axis and the drug delivery ports comprise a plurality of slits.

16. An implantable drug delivery system according to claim 13, wherein the drug delivery path preservation means comprises a means for delivering a substance for resisting fibrous occlusions through the drug delivery ports.

17. An implantable drug delivery system according to claim 13, wherein the drug delivery path preservation means comprises a fluid coating on the catheter.

18. An implantable drug delivery system according to claim 13, wherein the drug delivery path preservation means comprises a film on the catheter.

19. A method of treating erectile dysfunction comprising the steps of:
implanting a supply of prostaglandin E1 in the body in a device capable of releasing a dose on demand, and
thereafter treating the erectile dysfunction by releasing an effective amount of prostaglandin E1 on demand of the patient.

20. A method of treating erectile dysfunction according to claim 17 further comprising resisting chances of an overdose of the prostaglandin E1 by preventing actuation of the device outside predetermined parameters.
